(19) United States
(12) Patent Application Publication
(10) Pub. No.: US 2025/0152505 A1
(43) Pub. Date: May 15, 2025

(54) SPRAY COMPOSITIONS AND METHODS OF USE

(71) Applicant: NOOR BRANDS COMPANY, LLC, High Point, NC (US)

(72) Inventors: Adnan Mjalli, High Point, NC (US); Wiley William Hitchcock, High Point, NC (US)

(21) Appl. No.: 18/728,512

(22) PCT Filed: Jan. 13, 2023
(86) PCT No.: PCT/US2023/010772

§ 371 Date: Jul. 12, 2024

Related U.S. Application Data
(60) Provisional application No. 63/299,237, filed on Jan. 13, 2022.

(51) Int. Cl. A61K 9/00; A61K 9/12; A61K 31/122; A61K 36/71; A61K 47/02; A61K 47/10; A61K 47/26

(52) U.S. Cl. CPC A61K 9/0043; A61K 9/0073; A61K 9/12; A61K 31/122; A61K 36/71; A61K 47/02; A61K 47/10; A61K 47/26

(57) ABSTRACT

Disclosed herein are formulations of a spray comprising black seed oil (BSO). The spray can be used to treat and prevent diseases and disorders with pathologies involving the dysregulation of signaling pathways related to oxidative stress and inflammation. Formulations of the BSO spray can be delivered using nasal spray applicators, inhalers, and nebulizers.

### RELATED APPLICATIONS

This application claims priority to U.S. Provisional Application No. 63/299,237 filed Jan. 13, 2021. The disclosure of U.S. Provisional Application No. 63/299,237 is incorporated by reference in its entirety herein.

### FIELD

This present disclosure relates to compositions and methods of use for preventing or reducing the severity of inflammation in a subject. In certain embodiments, the compositions are spray formulations comprising black seed oil (BSO).

### BACKGROUND

Nigella sativa is a ubiquitous plant found throughout the Middle East. The seeds and oil from the Nigella sativa plant have been used in traditional medicine throughout Africa, Asia, and the Middle East as an herbal therapy for respiratory, cardiovascular, skin, renal, and gastrointestinal (GI) illness. Nigella sativa oil (NSO) (also known as black seed oil (BSO) or black cumin oil (BCO)) has been demonstrated to have anti-microbial, analgesic, and anti-inflammatory properties. The major bioactive compounds present in Nigella sativa include thymoquinone (TQ), thymol, limonene, and thymohydroquinone (Vanamala, J., et al. Plant Foods Hum Nutr. 67, 111-119 (2012)). Nigellone, a polymer of thymoquinone is also present in BSO.

Volatile oils of Nigella sativa (VONS) can be harvested from the seeds of the Nigella sativa plant. VONS have been demonstrated to have pharmacological activity. The phytochemical profile of VONS has been demonstrated to be rich in bioactive compounds, including high levels of monoterpenoids (˜50%), phytosterols, and alkaloids such as nigellamine. For example, the monoterpenoid ketone thymoquinone (˜20% of VONS) has been shown to have anti-oxidant, anti-microbial, anti-inflammatory, and analgesic properties.

However, delivery of the active ingredients in BSO to regions of interest in the body is challenging. The anatomy and physiology of the nasal cavity provide certain advantages for accessing for local, systemic, and central nervous system drug delivery. Thus, there is a need for formulations of BSO that can be delivered using spray and/or nebulizer platforms.

### SUMMARY

In one aspect, embodiments of the present disclosure relate to compositions comprising black seed oil (BSO). Disclosed are stable, functional, and safe spray formulations that are useful in mediating pro-inflammatory pathways and reducing tor preventing he severity of inflammation.

In certain embodiments, the total level of TQ in the final spray formulation of the composition is less than 0.1, 0.09, 0.08, 0.07, 0.06, 0.05, 0.04, 0.03, 0.02, 0.01, 0.009, 0.008, 0.007, 0.006, or 0.005%. In certain embodiments, the spray formulation comprises about 0.008-0.010% thymoquinone (TQ) total composition by volume. Or, other ranges as disclosed herein may be used. TQ is one of the major bioactive compounds of BSO and has demonstrated activity against a number of critical cellular pathways, including the NF-κB, Nrf2, P38, JNK, and ERK.

In a second aspect, embodiments of the present disclosure relate to methods of using the compositions disclosed herein. In some embodiments, the disclosure relates to methods of reducing the severity of inflammation in a subject comprising: (a) selecting a subject with inflammation or at risk for inflammation; and (b) administering to the subject a spray composition comprising the BSO. In certain embodiments the inflammation is associated with an infection, including but not limited to viral and bacterial infections. For example, the inflammation may be associated from an infection caused by SARS-COV-2 virus.

In another aspect, the disclosure provides a device for delivering a spray comprising BSO. In some embodiments, the spray can be delivered using a device. In certain embodiments the device is a nasal spray applicator, inhaler, or nebulizer.

### BRIEF DESCRIPTION OF THE DRAWINGS

The present disclosure may be better understood by referring to the following non-limiting figures.

FIG. 1 depicts aspects of conditions that may be treated, inhibited or amelioratied by Nigella sativa in accordance with an embodiment of the disclosure

FIG. 2 depicts the steps for isolating volatile black seed oil (BSO) from the seeds of the Nigella sativa plants in accordance with an embodiment of the disclosure.

FIG. 3 depicts the chemical structure of four of the major bioactive compounds present in Nigella sativa: thymoquinone (I), thymol (II), limonene (III) and thymohydroquinone (IV).

FIG. 4 depicts the chemical structure of nigellone, a polymer of thymoquinone.

FIG. 5 depicts induction of Nrf2 in response to TQ and the anti-inflammatory effects of TQ inhibition of NF-κB, P38, JNK, and ERK in accordance with an embodiment of the disclosure.

FIG. 6 depicts the regulation of oxidation and reactive oxygen species (ROS) by the Nrf2 signaling pathway in accordance with an embodiment of the disclosure.

FIG. 7 depicts characterizing some biological activities of Nrf2 activity in accordance with an embodiment of the disclosure.

### DETAILED DESCRIPTION

The following description recites various aspects and embodiments of the present compositions and methods. No particular embodiment is intended to define the scope of the compositions and methods. Rather, the embodiments merely provide non-limiting examples of various methods and systems that are at least included within the scope of the compositions and methods. The description is to be read from the perspective of one of ordinary skill in the art; therefore, information well known to the skilled artisan is not necessarily included.

### Definitions

The present disclosure now will be described more fully hereinafter. The disclosure may be embodied in many different forms and should not be construed as limited to the aspects set forth herein; rather, these aspects are provided so that this disclosure will satisfy applicable legal requirements. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

When introducing elements of the present disclosure or the embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements. It is understood that aspects and embodiments of the disclosure described herein include “consisting” and/or “consisting essentially of” aspects and embodiments.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B, and/or C” is intended to mean A alone, B alone, C alone, A and B in combination, A and C in combination, B and C in combination, or A, B, and C in combination.

Various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The term “subject” means an individual. For example, the subject is a mammal, such as a primate, and, more specifically, a human. Non-human primates can be subjects according to the present disclosure as well. The term “subject” includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical uses and formulations for non-humans are contemplated herein. The term does not denote a particular age or sex. Thus, adult and newborn subjects, whether male or female, are intended to be covered. As used herein, patient or subject may be used interchangeably and can refer to a subject afflicted with a disease or disorder.

The terms “treatment,” “treat,” “treating” and the related terms and expressions refer to reducing one or more of the effects (i.e., symptoms) of a disease or condition of a subject (e.g., one or more symptoms of a coronavirus infection or of cancer) by eliciting an immune response in the subject. Thus in the disclosed method, treatment can refer to a 10%, 20%, 30%, 40%, 50%, 60%, 70%, 80%, 90%, or 100% reduction in the severity of an established symptom or measurement of the disease or condition. For example, a method for treating a disease or condition is considered to be a treatment if there is a 10% reduction in one or more symptoms of the disease or condition in a subject, as compared to a control subject (e.g., an untreated subject). Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease or condition in the subject. Treating also refers to an action, for example, administration of a composition that occurs before or at about the same time a subject begins to show one or more symptoms of the condition or disease, which inhibits or delays onset or exacerbation or delays recurrence of one or more symptoms of the infection. As used in the present disclosure, references to decreasing, reducing, or inhibiting include a change of 10%, 20%, 30%, 40%, 50%, 60%, 70%, 80%, 90% or greater in the subject as compared to a control level. Thus, the reduction in onset, exacerbation or recurrence of the disease or condition can be about a 10, 20, 30, 40, 50, 60, 70, 80, 90, 100%, or any amount of reduction in between as compared to control subjects.

The terms “higher,” “increases,” “elevates,” or “elevation” refer to increases above basal levels, or as compared to a control. The terms “low,” “lower,” “inhibits,” “inhibition,” “reduces,” or “reduction” refer to decreases below basal levels, or as compared to a control. For example, basal levels are normal in vivo levels prior to, or in the absence of, inflammation or the addition of an agent which causes inflammation.

The term “mediate” or “mediation” and “modulate” or “modulation” means to regulate, or control, in particular to increase, enhance, elevate, or alternatively to lower, inhibit, or reduce. The terms “mediate” and “modulate” are used interchangeably throughout.

“Inflammation” or “inflammatory” is defined as the reaction of living tissues to injury, infection, or irritation. Anything that stimulates an inflammatory response is said to be inflammatory.

“Inflammatory disease” is defined as any disease state associated with inflammation. Examples of inflammatory disease include, but are not limited to, asthma, systemic lupus erythematosus, rheumatoid arthritis, reactive arthritis, spondyarthritis, systemic vasculitis, insulin dependent diabetes mellitus, multiple sclerosis, experimental allergic encephalomyelitis, Sjögren's syndrome, graft versus host disease, inflammatory bowel disease including Crohn's disease, ulcerative colitis, and scleroderma. Inflammatory diseases also includes autoimmune diseases such as myasthenia gravis, Guillain-Barré disease, primary biliary cirrhosis, hepatitis, hemolytic anemia, uveitis, Grave's disease, pernicious anemia, thrombocytopenia, Hashimoto's thyroiditis, oophoritis, orchitis, adrenal gland diseases, anti-phospholipid syndrome, Wegener's granulomatosis, Behcet's disease, polymyositis, dermatomyositis, multiple sclerosis, vitiligo, ankylosing spondylitis, Pemphigus vulgaris, psoriasis, dermatitis herpetiformis, Addison's disease, Goodpasture's syndrome, Basedow's disease, thrombopenia purpura, allergy; and cardiomyopathy.

“Infection” or “infectious process” is defined as one organism being invaded by any type of foreign material or another organism. The results of an infection can include growth of the foreign organism, the production of toxins, and damage to the host organism. Infection includes viral, bacterial, parasitic, and fungal infections, for example.

### Compositions

In one aspect, embodiments of the present disclosure relate to compositions comprising black seed oil (BSO). The present disclosure may be embodied in a variety of ways.

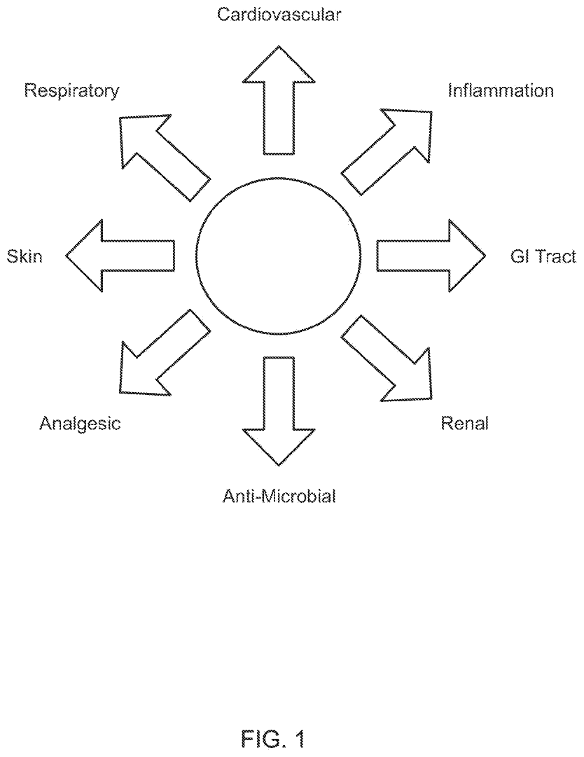

In one embodiment, disclosed is a composition comprising BSO derived from a Nigella sativa plant. Nigella sativa is a ubiquitous plant found throughout the Middle East. The seeds and oil from the Nigella sativa plant have been used in traditional medicine throughout Africa, Asia, and the Middle East as an herbal therapy for respiratory, cardiovascular, skin, renal, and gastrointestinal (GI) illness (FIG. 1). Nigella sativa oil (NSO) (also known as black seed oil (BSO) or black cumin oil (BCO)) has been demonstrated to have anti-microbial, analgesic, and anti-inflammatory properties.

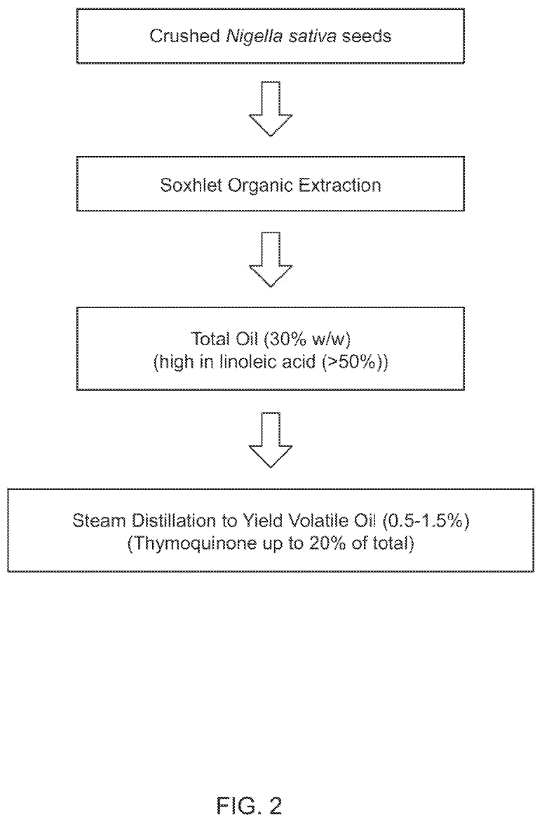

As depicted in FIG. 2, in certain embodiments, BSO can be produced by crushing seeds from Nigella sativa plants. Soxhlet organic extraction, which is commonly used for extracting bioactive compounds from natural sources, can then be used to produce total oil (30% w/w) from the crushed Nigella sativa seeds. The total oil produced from Soxhlet extraction of crushed Nigella sativa seeds is generally high in linoleic acid (>50%).

Following Soxhlet extraction, in certain embodiments, distillation can be used to yield volatile oils of Nigella sativa (VONS). Steam distillation is the most common method for producing oils from plants. However, the heat used during steam distillation can diminish the bioactive compounds present in the plant. Alternatively, pressed oils can be separated from the plant through mechanical means, without the use of heat. Thus, in some embodiments, the BSO is a cold pressed oil.

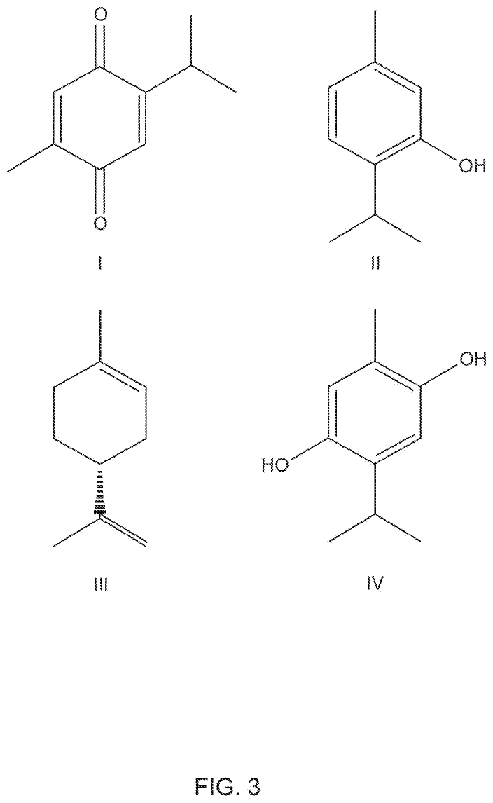

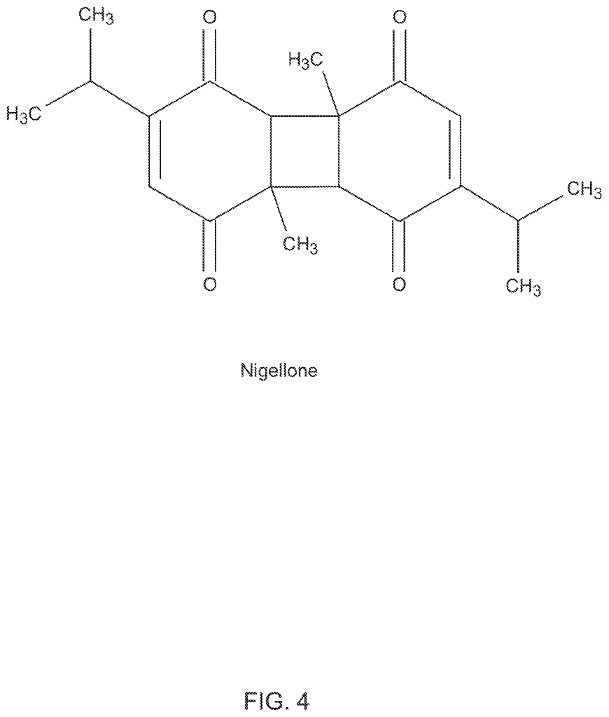

The phytochemical profile of VONS has been demonstrated to be rich in bioactive compounds, including high level of monoterpenoids (˜50% of VONS), phytosterols, and alkaloids such as nigellamine. For example, the monoterpenoid ketone thymoquinone (TQ) has been shown to have anti-oxidant, anti-microbial, anti-inflammatory, and analgesic properties. The major bioactive compounds present in Nigella sativa include thymoquinone (TQ), thymol, limonene, and thymohydroquinone (Vanamala, J., et al. Plant Foods Hum Nutr. 67, 111-119 (2012)). The structures of thymoquinone (I), thymol (II), limonene (III) and thymohydroquinone (IV) are depicted in FIG. 3. Nigellone, a polymer of thymoquinone, is also present in BSO. The structure of nigellone is depicted in FIG. 4.

In some embodiments, the composition comprises at least 0.05, 0.1, 0.25, 0.5, 0.75, 1, 1.25, 1.5, 1.75, 2, 2.5, or 3% BSO. The TQ content of BSO may vary depending on the methods of extraction from the Nigella sativa plant. In some embodiments, BSO may be obtained from a commercial supplier. For example, BIONATAL® Ethiopian BSO contains 4.34% thymoquinone (TQ). However, different methods produced BSO comprising between 0.9-2.6% TQ.

In some embodiments, TQ content can be used to determine a suitable concentration of BSO. In certain embodiments, the total level of TQ in the final formulation of the composition is less than 0.1, 0.09, 0.08, 0.07, 0.06, 0.05, 0.04, 0.03, 0.02, 0.01, 0.009, 0.008, 0.007, 0.006, or 0.005%. In some instances, the total level of TQ in the final formulation of the composition is between 0.008-0.01%. Or, other ranges as disclosed herein may be used.

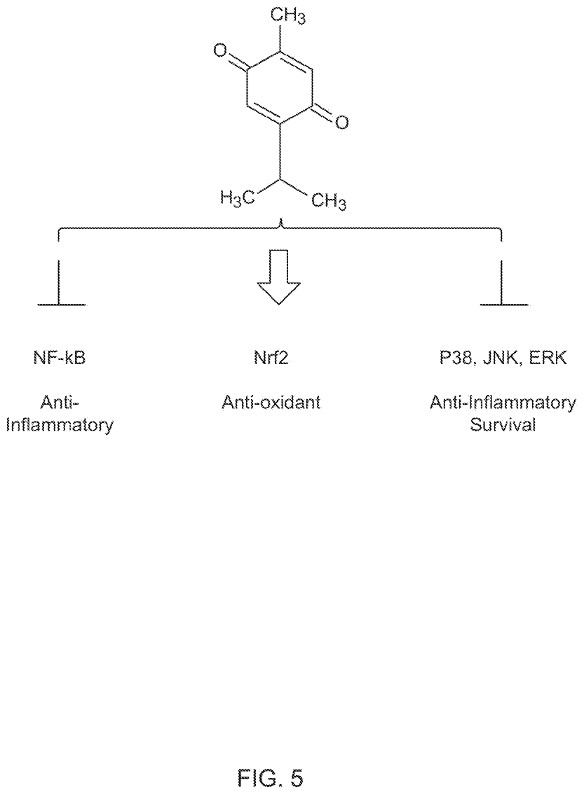

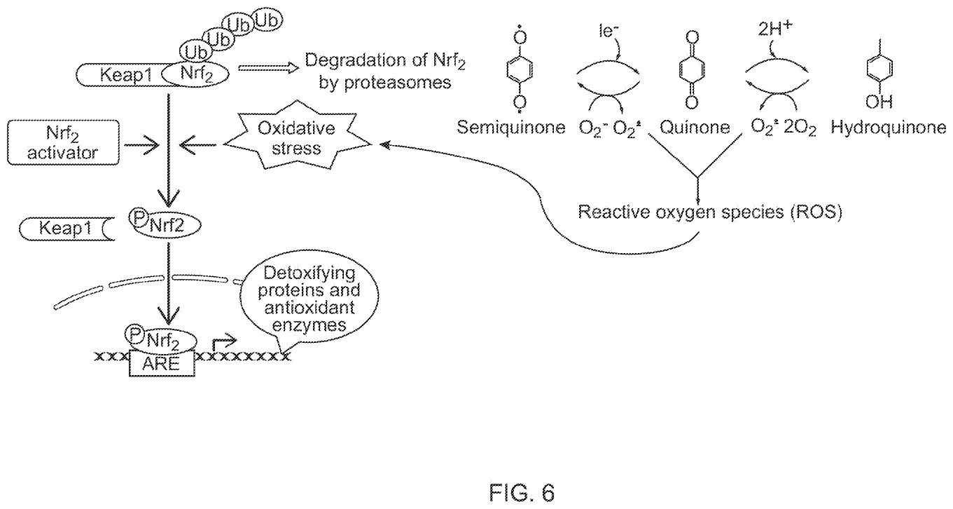

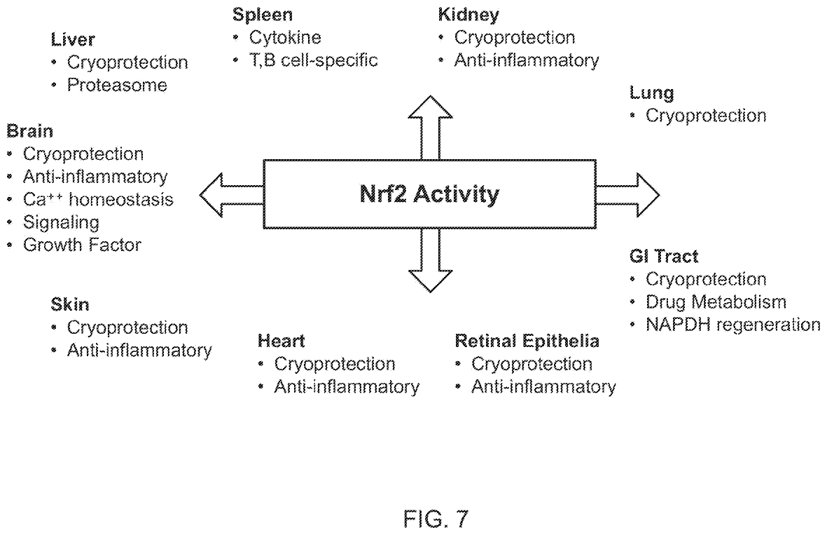

As shown in FIG. 5, in certain embodiments, TQ has activity against a number of critical cellular pathways, including the NF-κB, Nrf2, P38, JNK, and ERK. The NF-κB signaling pathway mediates inflammation. The Nrf2 signaling pathway may regulate oxidation as shown in FIG. 6. There are over 200 cytoprotective genes activated by Nrf2 and regulation of the Nrf2 pathway may have broad therapeutic activity including but not limited to activities in the liver (cryoprotection, proteasome), spleen (cytokine, T,B cell-specific), kidney (cryoprotection, anti-inflammatory), lung (cryoprotection), GI tract (cryoprotection, drug metabolism, NAPDH regeneration), retinal epithelia (cryoprotection, anti-inflammatory), heart (cryoprotection, anti-inflammatory), skin (cryoprotection, anti-inflammatory), and brain (cryoprotection, anti-inflammatory, Ca⁺⁺ homeostasis, signaling, growth factor) (FIG. 7). The P38, JNK, and ERK pathways mediate inflammation and survival.

In certain embodiments, the composition may comprise BSO in a pharmaceutically acceptable carrier. By “pharmaceutically acceptable” is meant a material that is not biologically or otherwise undesirable, i.e., the material may be administered to a subject, along with the BSO, without causing any undesirable biological effects or interacting in a deleterious manner with any of the other components of the pharmaceutical composition in which it is contained. The carrier would naturally be selected to minimize any degradation of the active ingredient (BSO) and to minimize any adverse side effects in the subject, as would be well known to one of skill in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

In some embodiments the composition comprises a pharmaceutically acceptable carrier. In certain instances the carrier comprises one or more of a surfactant, glycerol, polyethylene glycol, NaCl, and diH₂O.

In some embodiments, the composition may comprise one or more surfactants. Suitable surfactants may include polyoxyethylene sorbitan alkyl esters, and/or Polysorbates 20, 21, 40, 60, 65, 80, 81, 85. For example, the surfactant may be Polysorbate-80 (Polyoxyethylene (20) sorbitan monooleate, Tween 80, CAS #9005-65-6, FW 604.80). In some embodiments, the composition may comprise a quantity of Polysorbate-80 that ranges from about 1.0-2.0% of the total composition by volume. In some instances, the composition comprises a quantity of Polysorbate-80 that is about 0.1, 0.5, 1.0, 1.5, 2.0, 2.5, 3.0, 3.5, 4.0, 4.5, 5.0, 7.5, or 10% of the total composition by volume.

In some embodiments, the composition may further comprise glycerol. For example, the glycerol may be glycerol (CAS #56-81-5, FW 92.09). In some embodiments, the composition may comprise a quantity of glycerol that is about 1.5% of the total composition by volume. In some instances, the composition comprises a quantity of glycerol that is about 0.5,1.0, 1.5, 2.0, 2.5, 3.0, 3.5, 4.0, 4.5, or 5.0% of the total composition by volume.

In some embodiments, the composition may comprise one or more polyethylene glycols. Suitable polyethylene glycols may include, but are not limited to PEG-300 and PEG-400. For example, the polyethylene glycol may be PEG-400 (Polyethylene glycol (400) monolaurate), CAS #25322-68-3, FW 380-420). In some embodiments, the composition may comprise a quantity of PEG-400 that ranges from about 0.5-10.0% of the total composition by volume. In some instances, the composition comprises a quantity of PEG-400 that is about 0.1, 0.5, 1.0, 1.5, 2.0, 2.5, 3.0, 3.5, 4.0, 4.5, 5.0, 7.5, or 10% of the total composition by volume.

In some instances, the composition is isotonic. In certain embodiments, “isotonic” represents using 0.9% NaCl water, which must be added as the final step of the formulation process so that the emulsion will be stable. In some embodiments, the composition further comprises 0.9% NaCl diH₂O. In some instances, the composition comprises a quantity of 0.9% NaCl and diH₂O that is about 85, 86, 87, 88, 89, 90, 91, 92, 93, 94, 95, 96, 97, 98, or 99% of the total composition by volume.

In some embodiments, the suitable carrier may comprise one or more of: Polysorbate-80, glycerol, PEG-400, diH₂O, and/or NaCl.

In some embodiments, the composition comprises a spray formulation. In certain embodiments, the spray formulation comprises formulation #NBC-BSO47-Isotonic @ 1% BSO loading. In an embodiment, formulation #NBC-BSO47-Isotonic @ 1% BSO loading may comprise:

{(1% BSO (0.9% TQ)+2% Polysorbate-80+1.5% Glycerol+5% PEG-400+89.6% diH2O)+0.9% NaCl}.

In another embodiment, the spray formulation comprises formulation #NBC-BSO69-Isotonic @ 0.2% BSO loading. In an embodiment, formulation #NBC-BSO69-Isotonic @ 0.2% BSO loading may comprise:

{(0.2% BIONATAL® Ethiopian BSO (4.34% TQ)+1.5% Glycerol+1.0% Polysorbate-80+0.5% PEG-400) 95.9% diH2O+0.9% NaCl}

In some embodiments the composition is a spray. In certain embodiments, the spray is capable of being delivered nasally. The nasal cavity provides several advantages for accessing targets for local and/or systemic delivery of an agent (e.g., BSO). However, nasal drug delivery can be associated with several disadvantages such as damage to the nasal mucosa caused by the ingredients in the nasal spray formulation. For example, high levels of TQ have been shown to irritate the nasal cavity. Furthermore, some active agents may not be functionally stable in nasal spray formulations. Surprisingly, the disclosed formulations are both functionally stable and deliverable through the nasal cavity without mild to no irritation.

In some instances, the compositions disclosed herein are of benefit to subjects who are experiencing inflammation or are at risk for inflammation.

### Methods and Devices for Use of BSO Compositions

In a second aspect, embodiments of the disclosure relate to methods and devices for using the BSO compositions of the present disclosure. In some embodiments, the methods and devices are useful for reducing the severity of or preventing inflammation in a subject. In one embodiment, a method of reducing the severity of or preventing inflammation in a subject comprises: (a) selecting a subject with inflammation or at risk for inflammation and (b) administering to the subject a composition comprising the compositions described herein.

Inflammation can be associated with a number of different diseases and disorders. Examples of inflammation include, but are not limited to, inflammation associated with an inflammatory disease or disorder, cancer, or an infectious process.

The inflammation can be associated with an inflammatory disease or disorder. Examples of inflammatory diseases or disorders include, but are not limited to, rheumatoid arthritis, multiple sclerosis, myocardial infarction, COPD, chronic nephritis, chronic hepatitis, chronic pancreatitis, Type 2 diabetes, systemic lupus erythematosus (SLE), Alzheimer's disease, Parkinson's disease (PD), or inflammatory bowel disease (IBD).

The inflammation can also be associated with cancer. Examples of types of cancer include, but are not limited to, lymphoma (Hodgkins and non-Hodgkins) B-cell lymphoma, T-cell lymphoma, leukemia such as myeloid leukemia and other types of leukemia, carcinoma, adenocarcinoma, sarcoma, glioma, blastoma, neuroblastoma, plasmacytoma, histiocytoma, melanoma, adenoma, myeloma, AIDS-related lymphoma or AIDS-related sarcoma, metastatic cancer, bladder cancer, brain cancer, nervous system cancer, squamous cell carcinoma of the head and neck, neuroblastoma, glioblastoma, ovarian cancer, skin cancer, liver cancer, squamous cell carcinomas of the mouth, throat, larynx, and lung, colon cancer, cervical cancer, breast cancer, cervical carcinoma, epithelial cancer, renal cancer, genitourinary cancer, pulmonary cancer, esophageal carcinoma, head and neck carcinoma, hematopoietic cancer, testicular cancer, colorectal cancer, prostatic cancer, and pancreatic cancer.

Inflammation can be caused by an infectious process in a subject. When the inflammation is associated with an infectious process, the infectious process can be associated with a viral infection. Examples of viral infections include, but are not limited to, influenza virus, herpes simplex virus (HSV), human immunodeficiency virus (HIV) type 1 (HIV-1), HIV-2 Group A, HIV-2 Group B, HIV-1 Group M, Hepatitis B, Hepatitis Delta, Ebola virus, Marburg virus, Cueva virus, West Nile Virus, Epstein-Barr Virus, Dengue Virus, adenovirus B, adenovirus C, adenovirus E, Virus, Parainfluenza Virus type 1, Parainfluenza Virus type 2, Parainfluenza Virus, Coronavirus, 229E, Coronavirus HKU1, Coronavirus OC43, Coronavirus NL63, SARS-COV, MERS-COV, or SARS-COV-2. In certain embodiments, the formulation may protect against various variants of SARS-COV-2.

When the inflammation is associated with an infectious process, the infectious process can be associated with a bacterial infection. The bacterial infection can be caused by either gram positive or gram negative bacterium. Examples of bacterial infections include, but are not limited to, Bordetella pertussis, mycobacterium tuberculosis (MTB), Staphylococcus aureus, Methicillin-Resistant Staphylococcus aureus (MRSA), Group A streptococcus, Group B streptococcus, Haemophilus parainfluenzae, or Klebsiella pneumoniae.

In some embodiments, the methods provided herein can be used to mediate the activity of pro-inflammatory cytokines, hormones or other biologically active agents. In recent years there has been an increasing understanding that the human organism responds in a very similar way to disorders of different natures, trauma, exposure to infectious or non-infectious pathogens, and the risks of disease through the release of a cascade inflammatory mediators, which produce a response proportional to the magnitude of the disorder, disease, or infection.

In some embodiments, use of the compositions described herein mediates the activity of cytokines. In certain embodiments, use of the compositions decreases the levels of pro-inflammatory cytokines. For example, pro-inflammatory cytokines include, but are not limited to tumor necrosis factor alpha (TNF-α), interleukin-1 beta (IL-1β), interleukin-6 (IL-6), interleukin-8 (IL-8), interleukin-10 (IL-10), interleukin-12 (IL-12), interferon gamma (IF-γ), CXCL10, MCP1, and MIP1α.

Tumor Necrosis Factor-α (TNF-α) plays a central role in the initiation and regulation of the cytokine cascade during an inflammatory response. It is produced as a 26 kDa membrane-bound precursor molecule that is cleaved by the TNF-α converting enzyme to produce a 17 kD active cytokine. In inflammatory states or in diseases, TNF-α, together with several other pro-inflammatory mediators and neurotoxic substances is produced in high concentrations. TNF-α is a potential biomarker for diseases such as multiple sclerosis, Parkinson's disease, rheumatoid arthritis and diabetic retinopathy. It is also involved in the induction of granulocyte and macrophage colony stimulating factor (GM-CSF), which is related to tumor growth and progression in several types of cancers. In some embodiments, the compositions described herein decrease levels of TNF-α in the subject.

Interleukin 6 (IL-6) is a pro-inflammatory cytokine that can be secreted by many types of cells through appropriate stimulation during infection, inflammation or cancer. IL-6 can be important for regulating the responses of type B and T cells, as well as coordinating the activity of the innate and adaptive immune system. IL-6 can be strongly induced during most, if not all, inflammatory, infectious and cancerous processes. Also, in sepsis, IL-6 levels can reach mg per mL levels, and high amounts of this cytokine in the brain lead to neurodegeneration. IL-6 is a promising indicator in the treatment of several diseases such as rheumatoid arthritis, Castleman's disease, heart diseases, sepsis and more recently in COVID-19. In some embodiments, use of the compositions described herein decreases levels of IL-6 in the subject.

Interleukin 1 beta (IL-1β) is a member of the IL-1 cytokine family, being rapidly generated and released by different types of immune and non-immune cells in response to inflammatory signals. This pro-inflammatory cytokine may be upregulated in diseases such as colon, breast, oral and skin cancer. The overproduction of this cytokine is also observed in patients suffering from epilepsy, stroke, Alzheimer's disease and other neurological disorders, as well as in autoimmune diseases, in which it is one of the main responsible for the activation of these diseases. It is more recently correlated with type 2 diabetes, being induced by itself. In some embodiments, use of the compositions described herein decreases levels of IL-1β in the subject.

In some embodiments, use of the compositions described herein mediates critical cellular pathways in the subject. As shown in FIG. 5, TQ has demonstrated activity against a number of critical cellular pathways, including the NF-κB, Nrf2, P38, JNK, and ERK. The NF-κB signaling pathway mediates inflammation. The Nrf2 signaling pathway regulates oxidation as shown in FIG. 6. There are over 200 cytoprotective genes activated by Nrf2 and regulation of the Nrf2 pathway has been shown to have broad therapeutic activity including but not limited to activities in the liver (cryoprotection, proteasome), spleen (cytokine, T,B cell-specific), kidney (cryoprotection, anti-inflammatory), lung (cryoprotection), GI tract (cryoprotection, drug metabolism, NAPDH regeneration), retinal epithelia (cryoprotection, anti-inflammatory), heart (cryoprotection, anti-inflammatory), skin (cryoprotection, anti-inflammatory), and brain (cryoprotection, anti-inflammatory, Ca⁺⁺ homeostasis, signaling, growth factor) (FIG. 7). The P38, JNK, and ERK pathways mediate inflammation and survival.

Additionally, hormones play a role in the inflammatory response. For example, GLP-1, a glucose-regulating hormone, is understood to have protective effects against inflammation, particularly in subjects with diabetes. In some embodiments, use of the described spray compositions increases the amount of GLP-1 in the subject.

In some embodiments, the compositions and formulations of the present disclosure can be administered via any of several routes of administration, including, but not limited to, nebulization/inhalation, orally, intravenously, intramuscularly, subcutaneously, or transdermally. Administration can be by oral inhalation, nasal inhalation, or intranasal mucosal administration. Administration by inhalant can be through the nose or mouth via delivery by spraying or droplet mechanism, for example, in the form of an aerosol. In some embodiments, administration is intramuscular or subcutaneous.

Delivery of the compositions disclosed herein can be used therapeutically in combination with a pharmaceutically acceptable carrier. Pharmaceutical carriers are known to those skilled in the art. These most typically would be standard carriers for administration of drugs to humans, including solutions such as sterile water, saline, and buffered solutions at physiological pH. The pharmaceutical composition may be administered in a number of ways depending on whether local or systemic treatment is desired, and on the area to be treated.

The dosage ranges for the administration of the agents disclosed herein are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any contraindications. Dosage can vary, and can be administered in one or more dose administrations daily, for one or several days.

Thus, the methods according to the embodiments of the present disclosure are useful for both prophylactic and therapeutic purposes. Methods of treating or preventing an inflammatory response in a subject, which include administering to a subject with an infection or susceptible to an infection an effective dose of compositions or formulations described herein are also included among the embodiments of the present disclosure. In some embodiments, a composition or formulation as described herein can be used alone or in combination with one or more therapeutic agents such as, for example, antiviral compounds for the treatment of a viral infection or disease. For prophylactic use, an effective amount of a composition or formulation described herein can be administered to a subject prior to onset of an infection (for example, before obvious signs of infection) or during early onset (for example, upon initial signs and symptoms of infection). Prophylactic administration can occur at several days to years prior to the manifestation of symptoms. Prophylactic administration can be used, for example, in the preventative treatment of subjects identified as having a predisposition to an infection (e.g., coronavirus infection). Therapeutic treatment involves administering to a subject a therapeutically effective amount of a composition described in the present disclosure after diagnosis or development of infection.

Methods of treating cancer in a subject, which include administering to a subject with cancer or susceptible to developing a cancer an effective dose of compositions described herein, are also included among the embodiments of the present disclosure. The provided compositions and formulations can be used alone or in combination with one or more therapeutic agents.

In some embodiments, the methods provided herein can further comprise administering to the subject one or more additional therapies. For example, in methods of treating an infection in a subject, suitable additional types of therapies include anti-viral agents and antibiotics. For example, in methods of treating cancer in a subject, suitable additional types of therapies include, but are not limited to, chemotherapy, immunotherapy, radiotherapy, hormone therapy, differentiating agents, and small-molecule drugs. One of skill in the art will readily be able to select an appropriate additional therapy.

In some embodiments, the spray formulations are delivered via a device. Thus, in another aspect, embodiments of the disclosure relate to a device comprising the compositions or formulations described herein. In some embodiments, the device comprises a nasal spray applicator. In some embodiments, the device comprises a nebulizer. In some embodiments, the device comprises an inhaler. In some embodiments, the device holds a volume of at least 1, 2, 3, 5, 10, 15, 20, 30, 50, 100, 200, or 500 mL of the composition. In certain embodiments, the devices uses cold mist.

### EXAMPLES

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter.

### Example 1. BSO Composition

Example 1 is a spray formulation of black seed oil (BSO) with 0.9% TQ.

### Materials:

Polysorbate-80 (Polyoxyethylene (20) sorbitan monooleate, Tween 80), CAS #9005-65-6, FW 604.80,
Glycerol, CAS #56-81-5, FW 92.09, GRAS
PEG-400 (Polyethylene glycol (400) monolaurate), CAS #25322-68-3, FW 380-420,
NaCl (Sodium Chloride), CAS #7647-14-5, FW 58.44, GRAS
BSO (Black Seed Oil) (0.9% TQ), ideally using virgin, cold-pressed, full-spectrum source.

{(1% BSO (0.9% TQ)+2% Polysorbate-80+1.5% Glycerol+5% PEG-400+89.6% diH2O)+0.9% NaCl}.

### Formulation #NBC-BSO47-Isotonic @ 1% BSO Loading:

{(1% BSO (0.9% TQ)+2% Polysorbate-80+1.5% Glycerol+5% PEG-400+89.6% diH2O)+0.9% NaCl}.

### Example 2. BSO Composition

Example 2 is a spray formulation of BSO with 4.34% TQ.

### Materials:

Polysorbate-80 (Polyoxyethylene (20) sorbitan monooleate, Tween 80), CAS #9005-65-6, FW 604.80,
Glycerol, CAS #56-81-5, FW 92.09, GRAS
PEG-400 (Polyethylene glycol (400) monolaurate), CAS #25322-68-3, FW 380-420,
NaCl (Sodium Chloride), CAS #7647-14-5, FW 58.44, GRAS
BSO (Black Seed Oil) BIONATAL® Ethiopian BSO (4.34% TQ)
   Formulation #NBC-BSO69-Isotonic @ 0.2% BSO loading:

{(0.2% BIONATAL® Ethiopian BSO (4.34% TQ)+1.5% Glycerol+1.0% Polysorbate-80+0.5% PEG-400) 95.9% diH2O+0.9% NaCl}

### Example 3

### Applications:

For Nasal spray and inhalation BSO applications:

Pre-clinical test participants were tested using 5 ml, 10 ml, or 20 ml common nasal sprayers. The corresponding volume of the formulations described in Examples 1 and 2 were added to the nasal sprayer. Pre-clinical test participants were instructed to use the nasal sprayers as recommended by the manufacturers to screen for sinus application of the formulations.

### Example 4: Active Ingredient (BSO) Validation

Black seed (Nigella sativa) oil was tested in vitro to investigate its potential health benefits. Briefly, a macrophage cell line was treated with various concentration of BSO from different sources, including BIONATAL® Ethiopian BSO (4.34% TQ)

Macrophage cell lines treated with BSO showed a decrease in the production of pro-inflammatory cytokines TNF-alpha and interleukin-6 in a macrophage cell line and an increase in GLP-1, a glucose-regulating hormone whose upregulation is considered desirable to treat patients with diabetes.

### Example 5—Illustrative Embodiments of Suitable Compositions, Methods, and Devices

As used below, any reference to methods or systems is understood as a reference to each of those methods or systems disjunctively (e.g., “Illustrative embodiment 1-4 is understood as illustrative embodiment 1, 2, 3, or 4.”).

Illustrative embodiment 1 is a composition comprising black seed oil (BSO), wherein the composition is a spray.

Illustrative embodiment 2 is the composition of any preceding or subsequent illustrative embodiment, wherein the spray comprises 0.008-0.010% thymoquinone (TQ) total composition by volume.

Illustrative embodiment 3 is the composition of any preceding or subsequent illustrative embodiment, wherein the spray comprises 1% BSO, 2% Polysorbate-80, 1.5% Glycerol, 5% PEG-40, 89.6% diH2O, and 0.9% NaCl, wherein the BSO comprises 0.9% TQ.

Illustrative embodiment 4 is the composition of any preceding or subsequent illustrative embodiment, wherein the spray comprises 0.2% BSO, 1.5% Glycerol, 1.0% Polysorbate-80, 0.5% PEG-400, 95.9% diH2O, and 0.9% NaCl, wherein the BSO comprises 4.34% TQ.

Illustrative embodiment 5 is a method of reducing the severity of inflammation in a subject comprising:
a. selecting a subject with inflammation or at risk for inflammation and
b. administering to the subject a composition comprising the spray of any preceding or subsequent illustrative embodiment.

Illustrative embodiment 6 is the method of any preceding or subsequent illustrative embodiment, wherein the spray is administered within 24 hours before or after the subject is contacted with an inflammatory agent.

Illustrative embodiment 7 is the method of any preceding or subsequent illustrative embodiment, wherein the inflammation is associated with an infection.

Illustrative embodiment 8 is the method of any preceding or subsequent illustrative embodiment, wherein the infection is a viral infection.

Illustrative embodiment 9 is the method of any preceding or subsequent illustrative embodiment, wherein the infection is a bacterial infection.

Illustrative embodiment 10 is the method of any preceding or subsequent illustrative embodiment, wherein the viral infection is caused by a SARS-COV-2 virus.

Illustrative embodiment 11 is the method of any preceding or subsequent illustrative embodiment, wherein the spray mediates the activity of pro-inflammatory cytokines, hormones or other biologically active agents.

Illustrative embodiment 12 is the method of any preceding or subsequent illustrative embodiment, wherein the pro-inflammatory cytokines comprise at least one of TNF-alpha and interleukin-6.

Illustrative embodiment 13 is the method of any preceding or subsequent illustrative embodiment, wherein the hormone is GLP-1.

Illustrative embodiment 14 is the method of any preceding or subsequent illustrative embodiment, comprising application of the spray using at least one of a nasal spray applicator, a nebulizer, or an inhaler.

Illustrative embodiment 15 is a device comprising a spray of any preceding or subsequent illustrative embodiment.

Illustrative embodiment 16 is the device of any preceding or subsequent illustrative embodiment, comprising a nasal spray applicator.

Illustrative embodiment 17 is the device of any preceding or subsequent illustrative embodiment, comprising a nebulizer.

Illustrative embodiment 18 is the device of any preceding or subsequent illustrative embodiment, comprising an inhaler.

1. A composition comprising black seed oil (BSO), wherein the composition is a spray.

2. The spray of claim 1 comprising 0.008-0.010% thymoquinone (TQ) total composition by volume.

3. The spray of claim 1 comprising 1% BSO, 2% Polysorbate-80, 1.5% Glycerol, 5% PEG-40, 89.6% diH2O, and 0.9% NaCl, wherein the BSO comprises 0.9% TQ.

4. The spray of claim 1 comprising 0.2% BSO, 1.5% Glycerol, 1.0% Polysorbate-80, 0.5% PEG-400, 95.9% diH2O, and 0.9% NaCl, wherein the BSO comprises 4.34% TQ.

5. A method of reducing the severity of inflammation in a subject comprising:
(a) selecting a subject with inflammation or at risk for inflammation and
(b) administering to the subject a composition comprising the a spray comprising blackseed oil (BSO).

6. The method of claim 5, wherein the spray is administered within 24 hours before or after the subject is contacted with an inflammatory agent.

7. The method of claim 5, wherein the inflammation is associated with an infection.

8. The method of claim 7, wherein the infection is a viral infection.

9. The method of claim 7, wherein the infection is a bacterial infection.

10. The method of claim 8, wherein the viral infection is caused by a SARS-COV-2 virus.

11. The method of claim 5, wherein the spray mediates the activity of pro-inflammatory cytokines, hormones or other biologically active agents.

12. The method of claim 11, wherein the pro-inflammatory cytokines comprise at least one of TNF-alpha and interleukin-6.

13. The method of claim 11, wherein the hormone is GLP-1.

14. The method of claim 4, comprising application of the spray using at least one of a nasal spray applicator, a nebulizer, or an inhaler.

15. A device comprising a spray comprising blackseed oil (BSO).

16. The device of claim 15, comprising a nasal spray applicator.

17. The device of claim 15, comprising a nebulizer.

18. The device of claim 15, comprising an inhaler.
